(19) United States
(12) Patent Application Publication
(10) Pub. No.: US 2024/0359979 A1
(43) Pub. Date: Oct. 31, 2024

(54) MAXIMIZING SYNGAS CARBON UTILIZATION AND CONVERSION TO BIOFUEL

(71) Applicant: ENERKEM INC., Montreal (CA)

(72) Inventors: Jean-Pierre CRETE, Montreal (CA); Xeniya SAVELYEVA, Montreal (CA); Jérémie GAGNON, Montreal (CA); Louis DENOMME, Montreal (CA); Maxime BANVILLE, Montreal (CA); Maxime FOUCAULT, Montreal (CA); Michel CHORNET, Montreal (CA)

(73) Assignee: ENERKEM INC., Montreal, QC (CA)

(21) Appl. No.: 18/687,162

(22) PCT Filed: Aug. 26, 2022
(86) PCT No.: PCT/CA2022/051292

§ 371 Date: Feb. 27, 2024

Related U.S. Application Data
(60) Provisional application No. 63/237,692, filed on Aug. 27, 2021.

(51) Int. Cl. C01B 3/52; C01B 3/02; C01B 32/40

(52) U.S. Cl. CPC C01B 3/52; C01B 3/02; C01B 32/40; C01B 2203/0415; C01B 2203/0475; C01B 2203/0485; C01B 2210/0064

(57) ABSTRACT

It is provided a process for optimizing syngas carbon utilisation and syngas purity from a varying scrubbed syngas source before feeding into a syngas conversion unit comprising the steps of feeding a scrubbed syngas into a purification unit comprising at least one absorption unit to remove CO₂ from the scrubbed syngas. producing a clean CO+H₂+CO₂ syngas stream and a CO₂ rich stream: and mixing the clean CO+H₂+CO₂ syngas stream with hydrogen producing a balanced syngas stream. wherein the balanced syngas stream meet the stoichiometric ratio and purity requirement of the syngas conversion unit.

### TECHNICAL FIELD

It is provided a process for optimizing syngas carbon utilisation, syngas purity and subsequent syngas conversion into downstream syngas conversion unit.

### BACKGROUND

Methanol production from syngas is a well-known technology. Traditionally, such syngas has been produced from coal gasification, natural gas reforming or other fossil fuel gasification or catalytic and/or thermal reforming.

Syngas have also been documented to be produced from similar gasification and/or reforming technology using bio carbonaceous feedstock, such as biomass (e.g. wood, agricultural residues, or fast growing crops), plastic, biomass rich residues and/or waste. Syngas production has been used to produce alcohol (such as methanol, ethanol, and propanol), drop in fuel (e.g. hydrocarbon) and/or other chemicals (e.g. acetic acid, acrylic acid, or methyl acetate).

Several carbonaceous solid or liquid feedstock gasification and/or reforming technology end up generating a crude syngas streams with an H₂/CO ratio lower than 2.0, which is required per stoichiometry for the production of methanol, alcohol and Fischer Tropsch. H₂/CO ratio generated from these processes are often below 1.5 and even as low as 0.7 and below.

In coal or liquid fossil fuel gasification and/or reforming plants producing a crude syngas with an H₂/CO lower than that required per the ratio derived from the stoichiometric reactions of the desired end product, a water gas shift reactor is typically included in the plant design to shift a portion of the excess CO into additional H₂ to rebalance the overall plant H₂/CO ratio (per reaction 1 below). This water gas shift reactor also generate CO₂ as a by-product. Since the overall plant has an excess of CO₂, a process unit is required for CO₂ removal. Feedstocks also typically contain sulfur which are converted into reduced sulfur species (H₂S, COS, etc.) in the gasification and/or reforming units, such typical plant also contains an acid gas removal (AGR) unit that both remove CO₂ and sulfur species. Sulfur species are contaminants or poisons for several syngas conversion catalysts and also are undesired in most final chemical and/or fuel products.

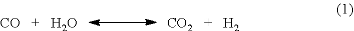

Depending on the specific catalyst and/or the desired final products, nitrogen contaminants such as HCN, NH₃ and/or amine must also be removed from the syngas. Such nitrogen contaminants can also be removed in the acid gas removal unit, with or without combination with other technology. Commercially available Cobalt based Fischer Tropsch catalyst is an example of catalyst that can only tolerate ultra-low level of such nitrogen contaminants; while also requiring to achieve ultralow sulfur contaminants level. Nitrogen contaminants removal from syngas can also benefit other syngas conversion technology, such as methanol or ethanol, to prevent trimethylamine (TMA) formation from the reaction of methanol with NH₃ and the subsequent additional purification effort to remove TMA from the final desired product.

In biomass, biomass rich or waste gasification and/or reforming valorization plant, such approach has the negative impact of losing valuable biogenic carbons via the carbon monoxide shift (equation 1), which does not end-up in the final biogenic product, but rather as excess CO₂ the plant has to either valorized as very low value merchant CO₂ and/or safely release to atmosphere after treatment and increase the green house impact of the plant. This also apply to plastic rich waste, waste (containing a significant non-bio fraction) or non-bio fossil derived waste gasification.

There is thus still a need to be provided with a process for minimizing lost in carbons (mostly as CO₂) and improving the yield via maximizing overall carbon syngas conversion, and by the same token the carbon feedstock conversion, to final desired product.

### SUMMARY

It is provided a process for optimizing syngas carbon utilisation, syngas purity and subsequent syngas conversion into a downstream syngas conversion unit from a scrubbed syngas source comprising the steps of feeding a scrubbed syngas into a purification unit comprising at least one absorption unit to partially remove CO₂ from said scrubbed syngas, producing a clean syngas stream and a CO₂ rich stream; mixing the clean syngas stream with hydrogen producing a balanced syngas stream, wherein the balanced syngas stream meet the stoichiometric ratio requirement of the syngas conversion unit; and feeding said balanced syngas stream in said syngas conversion unit.

In an embodiment, the at least one absorption unit further removes sulfur species and produces an additional sulfur species rich stream.

In a further embodiment, the sulfur species is H₂S, COS, CS₂, or a combination thereof.

In another embodiment, the at least one absorption unit further removes nitrogen contaminant species, which are combined with the sulfur species rich stream or produces an additional nitrogen contaminant rich stream.

In an embodiment, the varying scrubbed syngas source comprises between 25 to 45 mol % of H₂, 30 to 65 mol % of CO and 6 to 40 mol % of CO₂

In a further embodiment, the clean syngas stream composition comprises 30% to 50 mol % H₂, 40 to 68 mol % CO and 0 to 25 mol % CO₂.

In a further embodiment, the carbon content (CO₂+CO) in the optimised carbon recovery balanced syngas allow to increase the scrubbed syngas carbon conversion to final desired product by more than 65%.

In an embodiment, the process described herein further comprises a step of recycling the CO₂ rich stream upstream of the purification unit, preferably into a gasification unit, for use as inerting gas, exporting said CO₂ rich stream for Carbon Capture and Storage (CCS), and/or producing saleable merchant CO₂ from said CO₂ rich stream.

In a particular embodiment, the purification unit is an acid gas removal unit (AGR) comprising a selective solvent for absorbing CO₂ and sulfur species from the scrubbed syngas in a absorption unit using said selective solvent, producing a loaded solvent and a clean syngas stream, withdrawing the loaded solvent through at least one recirculation loop, pre-flashing said loaded solvent at an intermediate pressure to recover absorbed H₂ and CO, producing a CO₂ stream rich in H₂ and CO and a flashed solvent steam, flashing the flashed solvent at lower pressure to recover CO₂, generating a non-flammable CO₂ rich stream and a second flashed solvent; and stripping the second flashed solvent in a first stripping unit to remove the sulfur species, producing a rich sulfur species stream and a clean solvent which is recycled back into the absorption unit.

In a further embodiment, a first portion of the loaded solvent is withdrawn through a first recirculation loop and a second portion of the loaded solvent is withdrawn through a second recirculation loop; the first portion of the loaded solvent is pre-flashed at an intermediate pressure recovering the H₂ and CO contained in the loaded solvent, producing a first CO₂ stream rich in H₂ and CO and a first flashed solvent, and the second portion of the loaded solvent is pre-flashed at an intermediate pressure recovering H₂ and CO contained in the loaded solvent, producing a second CO₂ stream rich in H₂ and CO and a second flashed solvent; the first flashed solvent is flashed at lower pressure generating a non-flammable CO₂ rich stream and a clean solvent which is recycled back into the absorption unit; the first and second CO₂ streams rich in H₂ and CO are recycled in the scrubbed syngas stream upstream of the absorption unit and the second flashed solvent is stripped in a stripping unit to remove sulfur contaminants (ex. H₂S, COS, etc), producing a H₂S rich stream and a clean solvent which is recycled back into the absorption unit.

In another embodiment, the purification unit is an acid gas removal unit (AGR) comprising a selective solvent for absorbing CO₂, sulfur species and nitrogen species from the scrubbed syngas in the absorption unit using said selective solvent, producing a loaded solvent; and a clean syngas stream; withdrawing the loaded solvent through at least one recirculation loop; pre-flashing said loaded solvent at an intermediate pressure to recover absorbed H₂ and CO, producing a CO₂ stream rich in H₂ and CO and a flashed solvent steam; flashing the flashed solvent at lower pressure to recover CO₂, generating a non-flammable CO₂ rich stream and a second flash solvent; and stripping the second flash solvent in a stripping unit to remove the sulfur species, and nitrogen species, producing a rich sulfur and nitrogen species stream and a clean solvent which is recycled back into the absorption unit.

In another embodiment, a first portion of the loaded solvent is withdrawn through a first recirculation loop, a second portion of loaded solvent is withdrawn through a second recirculation loop, and a third portion of loaded solvent is withdrawn through a third recirculation loop where; the first portion of the loaded solvent is pre-flashed at an intermediate pressure recovering the H₂ and CO contained in the loaded solvent, producing a first CO₂ stream rich in H₂ and CO and a first flashed solvent; and the second portion of the loaded solvent is pre-flashed at an intermediate pressure recovering H₂ and CO contained in the loaded solvent, producing a second CO₂ stream rich in H₂ and CO and a second flashed solvent; and optionally, the third portion of the loaded solvent is pre-flashed at an intermediate pressure recovering the H₂ and CO contained in the loaded solvent, producing a third CO₂ stream rich in H₂ and CO and a third flashed solvent; the first flashed solvent is flashed at lower pressure generating a non-flammable CO₂ rich stream and a clean solvent which is recycled back into the absorption unit; the first, second and optional third CO₂ streams rich in H₂ and CO are recycled in the scrubbed syngas stream upstream of the absorption unit; and the second and third flashed solvents are stripped in a stripping unit to remove the sulfur species, and nitrogen species, producing a rich sulfur and nitrogen species stream and a clean solvent which is recycled back into the absorption unit.

In a further embodiment, a third portion of the loaded solvent is withdrawn through a third recirculation loop. The third portion of the loaded solvent is pre-flashed at an intermediate pressure recovering H₂ and CO contained in the third loaded solvent, producing a third CO₂ stream rich in H₂ and CO and a third flashed solvent. The third CO₂ stream rich in H₂ and CO is recycled in the scrubbed syngas stream upstream of the absorption unit. The third flashed solvent is stripped in the same stripping unit as the second flashed solvent to remove NH₃ and HCN, producing a H₂S, COS, NH₃ and/or HCN (i.e syngas contaminants) rich stream and a combined clean solvent which is recycled back into the absorption unit.

In a further embodiment, the third flashed solvent is stripped in a second separate stripping unit to remove NH₃ and/or HCN, producing a NH₃ and/and HCN rich stream.

In an embodiment, the first and/or the second stripping units are a thermal stripper to remove the sulfur and/or nitrogen species, producing a rich sulfur species gas stream, and/or a rich nitrogen species gas stream and clean solvents, wherein the clean solvents are recycled back into the at least one absorption unit.

In an embodiment, the stripping unit is a thermal stripper to remove the sulfur contaminants, producing a rich H₂S and/or sulfur contaminants gas stream and a clean solvent, wherein the clean solvent is recycled back into the at least one absorption unit.

In an embodiment, the second stripping unit is a thermal stripper to remove the nitrogen contaminants, producing a rich NH₃ and/or HCN gas stream and a clean solvent, wherein the clean solvent is recycled back into the at least one absorption unit.

In another embodiment, the solvent loop flow rates and temperatures, and the pressures of the pre-flashing steps are adjusted to achieve the target CO₂ content in the final clean syngas stream.

In a further embodiment, the sulfur species is H₂S, COS, CS₂, or a combination thereof.

In a supplemental embodiment, the nitrogen contaminant species are HCN, NH₃, an amine, or a combination thereof.

In an embodiment, the scrubbed syngas sources have variables yield, flowrate and/or composition overtime.

In a further embodiment, the scrubbed syngas sources variability is due to the variable nature of heterogeneous waste biomass, waste and/or plastic waste feedstock.

In an embodiment, the clean solvent(s) is (are) cooled before being recycled back into the at least one absorption unit.

In another embodiment, the thermal stripper(s) comprises a column comprising a reboiler and a condenser.

In an embodiment, the non-flammable CO₂ rich stream is further used as inerting gas, recovered for Carbon Capture and Storage (CCS) and/or to produce saleable merchant CO₂.

In a supplemental embodiment, the process further comprises treating the clean syngas stream with at least one solid adsorbent bed before or after mixing the clean syngas stream with hydrogen.

In an embodiment, at least one adsorbent bed comprises an alumina based adsorbent for HCl and halogen removal, a ZnO based adsorbent for removal of HCl (and halogen) and H₂S, a Cu based adsorbent for removing COS, CS₂ and arsine, and an adsorbent for carbonyl removal.

In another embodiment, the absorption unit is a column comprising at least 3 mass transfer zone sections.

In further another embodiment, the absorption unit is a column comprising at least 4 mass transfer zone sections.

In a further embodiment, the mass transfer zone sections are comprised in separated columns.

In a further embodiment, the hydrogen is imported from an external source.

In an embodiment, the imported hydrogen is from a renewable source and/or a source of low carbon intensity.

In another embodiment, the imported hydrogen is from a water electrolysis with renewable power or low carbon intensity power, a biogas reforming or steam reforming, a low carbon intensity (CI) hydrogen source, or a low CI waste H₂ source.

In a particular embodiment, the purification unit or AGR comprises chilled methanol has a solvent.

In an embodiment, the pressure and temperature of pre-flashing is adjusted to achieve the target CO₂ content and recovery in the final clean syngas stream.

In a further embodiment, the balanced syngas stream meet the stoichiometric ratio requirement of the syngas conversion unit to produce fuel, a chemical, or a Fischer Tropsch product.

In another embodiment, the chemical or fuel is methanol or ethanol.

In an embodiment, the Fischer Tropsch product is diesel, kerosene, a jet fuel, or a naphtha or a mixture thereof.

In another embodiment, the clean syngas stream at the AGR achieves less than 100 ppbv, less than 10 ppbv, or alternatively less than 5 ppbv HCN and NH₃.

In an embodiment, the clean syngas stream at the AGR achieves less than 10 ppmv, less than 5 ppmv, less than 1 ppmv, or alternatively less than 0.1 ppmv of combined sulfur species.

In another embodiment, the clean syngas stream at the solid adsorbent bed achieves less than 10 ppbv, or alternatively less than 5 ppbv of sulfur species, of halogen species, of arsine, and/or of metal carbonyl.

In an embodiment, the halogen species are HCl, HF, HBr, or a combination thereof.

In a further embodiment, the metal is Ni, Fe, or a combination thereof.

In another embodiment, H₂S concentration in the clean syngas stream is adjusted to achieves a specific desired concentration, to meet the requirement of the downstream syngas conversion unit; while achieving low level of HCN and/or NH₃ concentration.

In another embodiment, the H₂S concentration in the clean syngas stream is maintained below 200 ppmv.

In another embodiment, the H₂S concentration in the clean syngas stream is maintained below 100 ppmv.

In another embodiment, the process described herein further includes a reverse water gas shift (RWGS) unit to convert a portion of the recovered CO₂ along with a portion of the imported H₂ to generate additional carbon monoxide prior to the syngas conversion unit.

In a further embodiment, the additional CO production being mixed with a portion of the clean syngas to generate a boosted CO clean syngas.

In another embodiment, the CO₂ is recycled back to a carbonaceous feedstock gasification and/or reforming unit to reduce the scrubbed syngas H₂/CO ratio, increasing the total CO yield and production, and generating a boosted CO scrubbed syngas and a boosted CO clean syngas.

In an embodiment, the boosted CO clean syngas is mixed with hydrogen producing a balanced syngas stream, wherein the balanced syngas stream meet the stoichiometric ratio requirement of the syngas conversion unit.

In another embodiment, the syngas conversion unit converts in-situ H₂+CO and not H₂+CO₂.

In another embodiment, the process described herein further comprises admixing an external source of CO₂ or CO₂ input from another process effluent into the clean syngas stream along with the external source of hydrogen to produce a balanced syngas further boosted in carbon that meet the stoichiometric ratio of the desired end product, thus further increasing the production of the desired end product.

In an embodiment, the purification unit allows to achieve greater than 99% H₂+CO recovery in the clean syngas.

In a further embodiment, the scrubbed syngas is from a gasification and/or reforming of a carbonaceous material. Carbonaceous material refer to any gas, liquid or solid that contain a “Carbon” atom. In most cases, these atoms may be originated from plants or animals and their derivatives, or from fossil fuel and its derivative. As example of materials include, but not limited to, Municipal Solid Waste (MSW); Industrial, Commercial, and Institutional waste (IC&I); Construction and Demolition waste (C&D); any petroleum product; plastic; homogenous and/or non-homogeneous biomass.

In a particular embodiment, the carbonaceous material comprises a plastic, a metal, an inorganic salt, an organic compound, industrial wastes, recycling facilities rejects, automobile fluff, municipal solid waste, ICI waste, C&D waste, refuse derived fuel (RDF), solid recovered fuel, sewage sludge, used electrical transmission pole, railroad ties, wood, tire, synthetic textile, carpet, synthetic rubber, materials of fossil fuel origin, expended polystyrene, poly-film floc, construction wood material, or any combination thereof. The method according to the invention is applicable in principle to any carbon-based material. It is immaterial here whether the fuel is a biogenic or non-biogenic fuel.

In a further embodiment, the carbonaceous material is a biomass, a biomass rich waste, a plastic rich waste or waste.

In another embodiment, the carbonaceous material is waste plastic, waste rubber, or tire rich waste feedstock.

In an embodiment, the additional source of CO₂, is recovered within the purification unit by adding a CO₂ recovery and purification unit to produce a high quality and/or ultra-clean CO₂ streams.

In a further embodiment, the CO₂ recovery and purification unit includes a primary and/or a secondary CO₂ columns, where medium quality CO₂ is produced in the primary CO₂ column, and/or high quality CO₂ is produced in the secondary CO₂ column.

In another embodiment, a portion or all of the CO₂ loaded solvent from the first recirculation loop is used as scrubbing medium in the primary and/or secondary CO₂ columns; the CO₂ and sulfur species loaded solvent from the second recirculation loop is fed at the bottom of the primary CO₂ column; CO₂ and sulfur species loaded solvent from the bottom of the primary CO₂ column is fed to the secondary CO₂ column; and the enriched sulfur species loaded solvent from the bottom of the secondary CO₂ column is fed to the stripping unit.

In an embodiment, the high quality CO₂ is further treated with a solid adsorbent to produce an ultra high CO₂ quality.

In an embodiment, the ultra high CO₂ quality is either admixed into the clean syngas stream along with the external source of hydrogen to produce a balanced syngas further boosted in carbon that meet the stoichiometric ratio of the desired end product, thus further increasing the production of the desired end product; sold off-site; and/or sent for storage.

In another embodiment, the produced ultra high CO₂ quality is sent, along with additional imported hydrogen, to a separate second syngas conversion unit that produces a desired end co-product from H₂ and CO₂.

In a further embodiment, the co-product syngas conversion unit is a methanol catalytic reactor, a Fischer Tropsch reactor using iron based catalyst, or an ethanol reactor using micro-organism bio-catalyst.

In an embodiment, the first syngas conversion unit can converts in-situ H₂ and CO and cannot convert H₂ and CO₂, into the desired end product.

In an embodiment, the purification unit comprises a stripping unit including a split loading stripper column with at least a top and bottom mass transfer zone sections, where nitrogen species loaded methanol is fed at the top of the stripper column and above the top mass transfer zone, while the sulfur species loaded methanol is fed in the middle of the stripper column in between the top and bottom mass transfer zone sections.

### BRIEF DESCRIPTION OF THE DRAWINGS

Reference will now be made to the accompanying drawings.

FIG. 1 illustrates a flow chart of the process described herein in accordance to an embodiment.

FIG. 2 illustrates a flow chart of the process described herein in accordance to an embodiment, including details of the purification unit.

FIG. 3 illustrates a flow chart of the process described herein in accordance to an embodiment, including details of the purification unit and the option to achieve high removal efficiency of HCN and/or NH₃.

FIG. 4 illustrates a flow chart of the process described herein in accordance to an embodiment, including details of the purification unit and the option to achieve high removal efficiency of HCN and/or NH₃ with a second stripping unit.

FIG. 5 illustrates a flow chart of the process described herein in accordance to an embodiment, including details of a CO₂ recovery and purification unit and an option to recover additional ultra-clean high quality CO₂ within the AGR. FIG. 5 is also showing a configuration where the fourth mass transfer zone for HCN/NH₃ removal is installed in a separate column.

FIG. 6 illustrates a flow chart of the process described herein in accordance to an embodiment, including details of a split loading nitrogen and sulfur species stripping unit, and option to further improve the efficiency of the AGR.

### DETAILED DESCRIPTION

In accordance with the present disclosure, it is provided a process for optimizing syngas carbon utilisation, syngas purity and subsequent syngas conversion into downstream syngas conversion unit.

It is provided a method for maximising yield of syngas derived product (e.g. methanol) from gasification and/or reforming of variable carbonaceous feedstock composition when an external source of hydrogen, preferably a green, renewable or low carbon intensity hydrogen, is available.

It has been disclosed that rather than shifting excess CO to H₂ in a plant using carbonaceous feedstock, an external source of hydrogen could be imported into the plant and combined with the plant rich CO syngas to rebalance the overall plant H₂/CO ratio to that required per the ratio derived from the stoichiometric reactions of the desired end product.

It is also known that some chemicals and fuel can be produced from the reaction of H₂ and CO, but also from H₂ and CO₂. One such product is methanol, but also Fischer Tropsch using iron based catalyst and ethanol using micro-organism bio-catalyst.

In most existing syngas to methanol plant, methanol is produced in a catalytic reactor per the following chemical reactions.

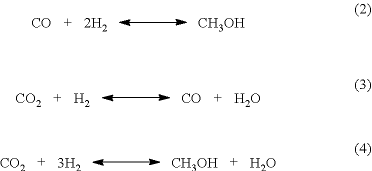

It is understood and required that a minimum amount of CO₂ is needed in the syngas to the methanol reactor to obtain high methanol productivity and higher dry basis methanol purity (i.e. kg/hr Methanol per kg catalyst).

Typical modern syngas to methanol plant prepares a make-up syngas to be sent to a methanol reactor loop. In order to achieve high carbon efficiency (CO and CO₂ conversion to methanol), the make-up syngas must be balanced., i.e. any large excess of CO, CO₂ or H₂ would results in losses of valuable molecules via the reactor loop purge gas stream, as inevitably some non-condensable gases that act as inert/diluent (usually N₂, CH₄, etc.) must be purged from the system.

A fully balanced syngas would have a stoichiometric ratio or number (SN) of 2.0, leading theoretically to full conversion of H₂, CO and CO₂ to methanol as per above three reactions. Typically, a slight excess of H₂ is recommended, resulting in an SN slightly above 2.

$$SN = \frac{( {H}_{2} - {CO}_{2} )}{( CO + {CO}_{2} )} \qquad ( 5 )$$

Since modern methanol reactor design and catalyst can also convert CO₂ with H₂ to methanol, carbonaceous rich feedstock gasification and/or reforming plants including an external source of hydrogen would benefit from a novel plant design as proposed herewith which would maximize carbonaceous feedstock derived CO and CO₂ recovery to achieve higher methanol yield.

Table 1 shows the range of CO₂ concentration in the make-up gas for a constant stoichiometric number (SN) of 2.04, ranging from no CO₂ (all methanol via equation 2) up to no CO (all methanol via net reaction 4). Accordingly, methanol can be produced from CO rich syngas to CO₂ rich syngas, and in any proportion in between.

TABLE 1
CO₂ Concentration in Methanol Reactor
Make-up Gas at constant SN

Syngas Composition (% v/v) - Main components

| | | | % Non-condensable | |
|---|---|---|---|---|
| | | | and others | |
| H₂ | CO | CO₂ | (N₂, CH₄, etc.) | SN |
| 66.0% | 32.3% | 0.0% | 1.8% | 2.04 |
| 66.9% | 28.3% | 3.0% | 1.8% | 2.04 |
| 67.5% | 25.7% | 5.0% | 1.8% | 2.04 |
| 68.2% | 23.0% | 7.0% | 1.8% | 2.04 |
| 69.2% | 19.0% | 10.0% | 1.8% | 2.04 |
| 70.0% | 15.7% | 12.5% | 1.8% | 2.04 |
| 70.8% | 12.4% | 15.0% | 1.8% | 2.04 |
| 71.7% | 9.1% | 17.5% | 1.8% | 2.04 |
| 72.5% | 5.7% | 20.0% | 1.8% | 2.04 |
| 73.9% | 0.0% | 24.3% | 1.8% | 2.04 |

One of the challenges of waste, plastic rich waste, biomass and/or biomass rich gasification and/or reforming is the feedstock gasification/reforming composition variability overtime (weekly, monthly variations due to the variable nature of such feedstock), which impact the H₂, CO, CO₂ yield (flow rate) and contaminants concentration in the produced scrubbed syngas, i.e. the “varying scrubbed syngas source”. The downstream units design, especially the purification unit, must then have the ability to manage variable syngas composition while maximizing CO and CO₂ recovery for maximum methanol or biofuel production while achieving target contaminants removal.

In an embodiment, the contaminants comprises, but are not limited to; sulfur species such as H₂S, COS and/or CS₂, nitrogen species such as HCN, NH₃ and/or amine, water and aromatics such as benzene, toluene and xylene. Nitrogen (N₂) is not a contaminant.

In a further embodiment, the purification unit is an acid gas removal unit (AGR) comprising a selective solvent for absorbing CO₂, sulfur contaminants (ex. H₂S, COS, and/or CS₂, etc), and nitrogen contaminants (ex. HCN, NH₃, and/or amine).

As described herein, the process provided for methanol production from a carbonaceous feedstock gasification and/or reforming also apply to other product which use methanol as intermediate and/or directly from syngas and/or other intermediate.

In an embodiment, the carbonaceous material/feedstock comprises a plastic, a metal, an inorganic salt, an organic compound, industrial wastes, recycling facilities rejects, automobile fluff, municipal solid waste, ICI waste, C&D waste, refuse derived fuel (RDF), solid recovered fuel, sewage sludge, used electrical transmission pole, railroad ties, wood, tire, synthetic textile, carpet, synthetic rubber, materials of fossil fuel origin, expended polystyrene, poly-film floc, construction wood material, or any combination thereof. Accordingly, it is encompassed a process for methanol production from a carbonaceous feedstock gasification and/or reforming, such as e.g. a biomass, a biomass rich waste, a plastic rich waste and/or waste.

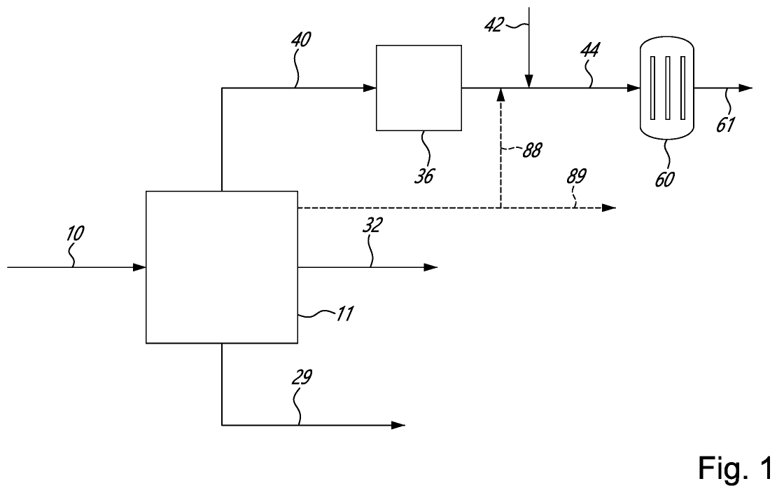

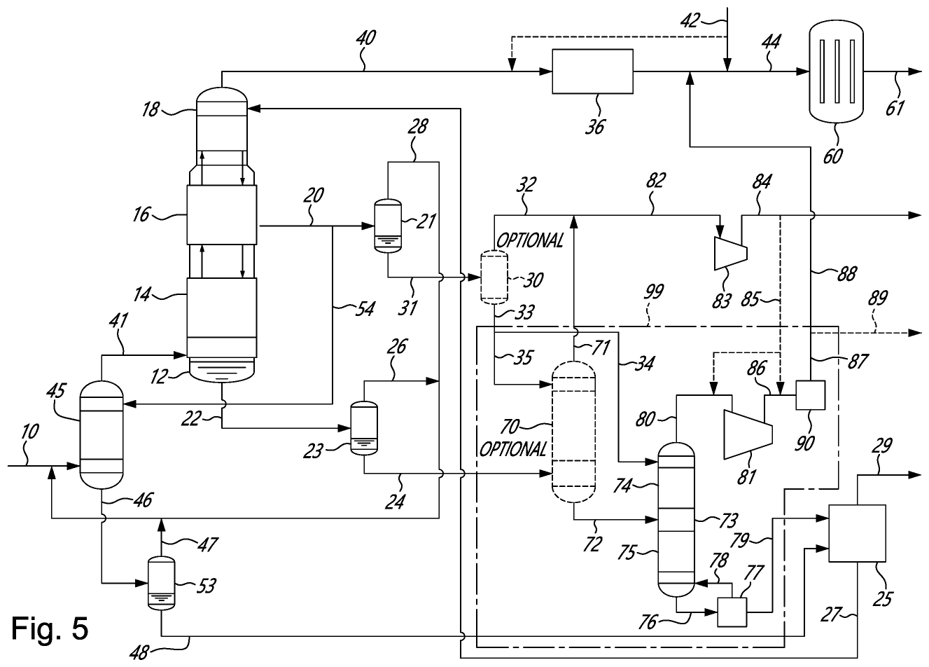

As depicted in FIG. 1, it is provided a process as described herein which involves maximizing the hydrogen, carbon monoxide and carbon dioxide recovery in the purification unit 11. Scrubbed syngas 10 is first feed to a purification unit 11 in order to optimize the CO₂ recovery in the purification unit 11 to maximise the CO₂ content in the clean syngas 40 leaving the purification unit 11. Imported hydrogen 42 is injected in the clean syngas 40 to prepare a balanced make-up syngas 44 to feed the catalytic syngas conversion unit 60 to produce the desired end product 61. A non-flammable CO₂ rich stream 32 is generated, which can be used as inerting gas up-stream in the plant. In addition, in an embodiment, a high quality CO₂ stream or ultraclean CO₂ stream (88) can be generated to optionally be mixed with the clean syngas stream 40 along with the external source of hydrogen 42 to produce a balanced syngas 44 further boosted in carbon that meet the stoichiometric ratio target for the desired end product, thus further increasing the production of the desired end product (see FIG. 5). Furthermore, in another embodiment, the ultraclean CO₂ can be sold off-site or send for storage (i.e. CCS) (stream 89). The process described herein allows removing the scrubbed syngas contaminants 29 in the purification unit 11.

Since the yield of H₂+CO over CO₂ in the varying scrubbed syngas source will vary over time, the AGR design and downstream syngas conversion unit must be designed to manage a range of syngas compositions, and must have the ability to adapt to such syngas yield variations while maximizing at all time the production within the plant and H₂ import capacity.

The syngas yield refer to the quantity of H₂, CO and CO₂ produced by the gasification/reforming unit per input quantity of carbonaceous feedstock fed to the gasification/reforming unit. Syngas yield variations imply that total syngas flowrate and composition, and thus individual H₂, CO and CO₂ flowrate, will vary overtime (weekly, monthly variations due to the variable nature of such feedstock).

Furthermore, downstream of the gasifier/reformer there are often steps of hot syngas quench, heat recovery and wet scrubbing using water as scrubbing medium. The wet scrubbed syngas then feed the AGR with or without a compression step. A COS hydrolysis to H₂S unit may also be included before the AGR, which also typically catalyse HCN hydrolysis to NH₃.

It is thus provided a novel AGR design for processing the scrubbed syngas through an acid gas removal unit specially designed to manage variable scrubbed syngas yield and split it into different gas streams:
(i) a clean syngas stream with low sulfur contaminants concentration (H₂S, COS, and/or CS₂, but not limited to), and optionally low nitrogen contaminants concentration (HCN and/or NH₃, but not limited to).
(ii) rich syngas contaminants loaded gas streams (with sulfur species and/or HCN/NH₃);
(iii) low quality CO₂ stream(s) rich in H₂ and CO (recycled back to the syngas inlet of the AGR absorption column);
(iv) a non-flammable rich CO₂ stream (medium quality CO₂); and
(v) optionally a high quality and/or ultra clean CO₂ stream.

The novel AGR design has operating handles that allow to maximise H₂ and CO recovery while adjusting the CO₂ recovery with variable inlet scrubbed syngas compositions to maximise methanol production.

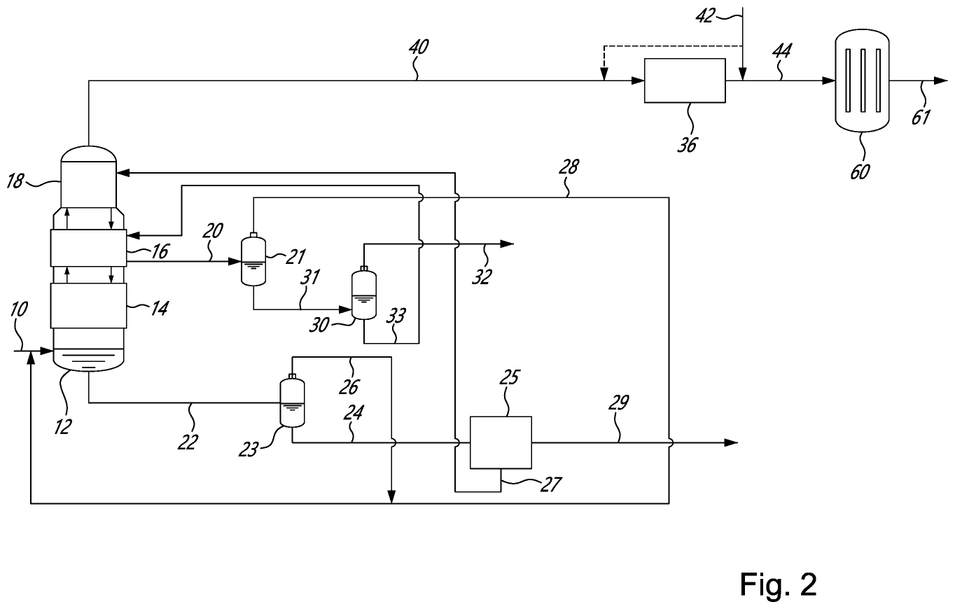

As illustrated in FIG. 2, the scrubbed syngas 10 is first feed to an absorption column 12 using a solvent selective for CO₂ and H₂S and other sulfur species. Such solvent can be for example but not limited to, chilled methanol.

The absorption column 12 has at least 3 mass transfer zone sections (example, but not limited to, trays, random packing and/or structured packing, namely the top 18, middle 16 and bottom section 14). The 3 mass transfer zone sections can be installed in one single column 12 with 3 sections or in 2 or 3 separate columns.

The loaded solvent still containing H₂ and CO passes through at least 2 recirculation loops around the absorber, the first loop 20 and the second loop 22. The second loop 22 is taken from the bottom section 14 and the first loop 20 is taken from the middle section 16 of the absorption column 12.

Both loops that withdraw solvent from the absorber sections, are pre-flashed (21, 23) at intermediate pressure to recover the valuable H₂ and CO absorbed in the solvent, thus producing two CO₂ streams rich in H₂ and CO—one 26 for the second loop 22 and one for the first loop 20 which are then combined and recycle up-stream of the absorption column 12, in the syngas feed 10. The recovered valuable H₂ and CO have another chance to pass through and leave the absorption column as part of the clean syngas 40 sent to the downstream catalytic reactor unit (60). Such pre-flash and recycle allow for more than 99% H₂+CO recovery in the overall AGR unit (i.e. Recovery=Quantity of H₂ and CO in stream 40 divided by the quantity of H₂ and CO in the inlet scrubbed syngas stream 10).

As a second step, the first loop solvent 20 is then further flashed 30 at lower pressure to generate a non-flammable CO₂ rich stream 32, which can be used as inerting gas up-stream in the plant. Alternatively, this CO₂ rich stream can be recovered for Carbon Capture and Storage (CCS) and/or to produce saleable merchant CO₂, with or without additional processing. The clean solvent is recycled back 33 into the absorption column 12.

As a second step, the pre-flashed second loop solvent 24 is then fed for regeneration to a thermal stripping unit 25 to remove the absorbed sulfur contaminants, thus producing a rich sulfur contaminants gas stream 29 and a clean regenerated solvent stream which is recycled back 27 at the top section 18 of the adsorber 12. The thermal stripping unit 25 can be a column with trays or packing equipped with either or both a reboiler and a condenser.

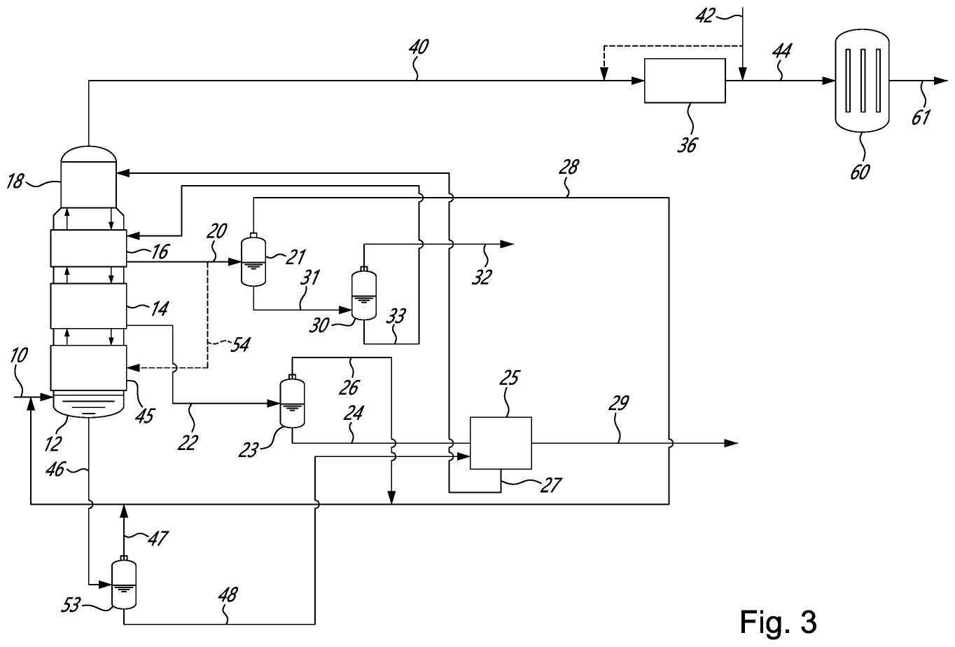

As illustrated in FIG. 3, when required the purification unit can be fitted with a third recirculation loop (46) to increase NH₃ and HCN removal efficiency. The fourth mass transfer zone section 45 can be added in the main absorption column 12 (FIG. 3.) or installed in a separate column (FIG. 5). The third loop solvent 46 is pre-flashed at intermediate pressure 53 to recover the valuable H₂ and CO absorbed in the solvent, thus producing a CO₂ stream rich in H₂ and CO 47 which is then combined and recycle up-stream of the absorption column 12, in the syngas feed 10.

The pre-flashed third loop solvent 48 is then fed for regeneration to the thermal stripping unit 25 to remove the absorbed nitrogen contaminants, thus producing a rich sulfur species, NH₃ and HCN gas stream 29.

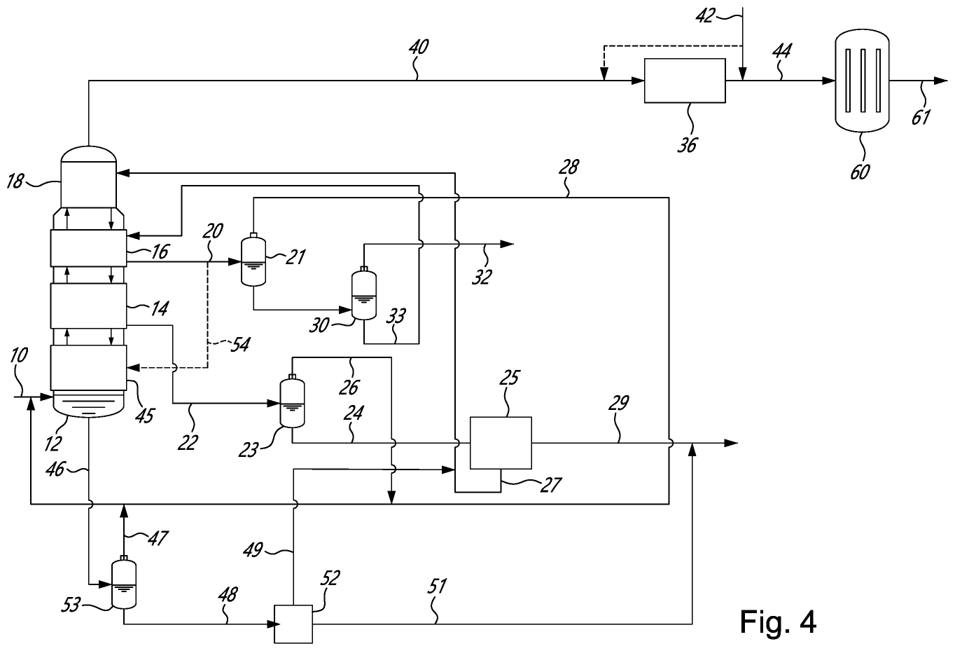

Alternatively, as illustrated in FIG. 4, the pre-flashed third loop solvent 48 can be fed to a separate thermal stripping unit 52 to remove absorbed nitrogen contaminants, thus producing a rich NH₃ and HCN gas stream 51 and a clean solvent 49.

Alternatively, the solvent fed to the fourth mass transfer zone section 45 can be supplied partially or entirely from a slip stream 54 from the first solvent loop 20, as shown on FIGS. 3 and 4. In this configuration, the second solvent loop 22 can thus be either a total or partial liquid draw from the third mass transfer zone 14. When the fourth mass transfer zone section 45 is installed in a separated column, as in FIG. 5,
the scrubbed syngas 10 is fed at the bottom of mass transfer zone section (53);
solvent stream 54 taken from the first recirculation loop 20 is fed at the top of the fourth mass transfer zone section 45, and
the pretreated gas 41 leaving the top of the fourth mass transfer zone section 45 is fed at the bottom of the absorber column 12 (bottom of the third mass transfer 14).

When low level of nitrogen species contaminants are present in the scrubbed syngas and thus requiring a lower third loop flowrate, the recoverable H₂+CO and CO₂ in the pre-flash step 53 will also be lower. As such, the extra project CAPEX to install step 53 may not be justified. In such a cases, the absorbed H₂+CO and CO₂ in third solvent loop 46 would be lost in the stripper vent stream 29 (FIG. 3) or 51 (FIG. 4).

The clean regenerated solvent 49 and 27 are taken respectively from stripping unit 52 (FIG. 4) and/or stripping unit 25 (FIGS. 2 and 3) and then cooled prior to be fed at the top section 18 of the adsorber 12 thus removing the final traces of sulfur and/or nitrogen contaminant from the syngas and thus producing a clean syngas stream 40 with low sulfur contaminants concentration (FIGS. 2, 3 and 4) and/or very low nitrogen contaminants concentration (FIGS. 3 and 4).

If required, to protect the downstream syngas conversion catalyst, the clean syngas stream 40 is further treated in a solid adsorbent bed 36 (before or after H₂ import addition 42), including a single or multiple adsorbent beds, in one or more parallel or/and in series vessel(s). Adsorbent may include alumina based adsorbent for removal of HCl and halogen, ZnO based adsorbent for removal of HCl, halogen and H₂S, a Cu based adsorbent for removing COS, CS₂ and Arsine, an adsorbent for carbonyl (Fe or Ni, or other) removal and/or adsorbent for HCN and/or NH₃ removal.

The clean syngas stream 40 is mixed with an external source of hydrogen 42 to produce a balanced syngas that meet the stoichiometric ratio requirement of the syngas conversion unit, thus producing an optimised carbon recovery balanced syngas 44.

The improved process of the present disclosure allow to produce an optimised carbon recovery balanced syngas which in turn allow to increase overall carbon scrubbed syngas and carbon feedstock conversion into the final desired product by more then 65% (as compared with the traditional approach of shifting the excess CO with water gas shift —WGS— and removing the excess CO₂), and even more then 140% depending on the initial scrubbed syngas composition (see Tables 2 and 3).

TABLE 2
Example composition of scrubbed syngas (10) and resulting
improved process clean syngas composition (40)

| | | | | | | Improved Process | | | |
|---|---|---|---|---|---|---|---|---|---|
| | | Scrubbed Syngas (10) | | | | Clean Syngas (40) | | | |
| Scrubbed Syngas Case (10) | | #1 | #2 | #3 | #4 | #1 | #2 | #3 | #4 |
| Concentration\Configuration | | — | — | — | — | B | B | B | B |
| H2 | mol % | 27% | 31% | 43% | 32% | 33% | 38% | 47% | 33% |
| CO | mol % | 40% | 41% | 43% | 62% | 49% | 49% | 47% | 65% |
| CO2 | mol % | 31% | 26% | 14% | 6% | 16% | 10% | 5% | 1% |
| N2 | mol % | 1% | 1% | 1% | 0% | 2% | 2% | 1% | 1% |

TABLE 3
Improved process scrubbed syngas carbon recovery with H₂ import compared to traditional approach with WGS.

| | | Scrubbed Syngas (10) | | | | Traditional Approach with | | | | Improved Process with H2 | | | |
|---|---|---|---|---|---|---|---|---|---|---|---|---|---|
| | | (Total 100 kmol/h basis) | | | | WGS - Balanced Syngas | | | | import - Balanced Syngas (44) | | | |
| Scrubbed Syngas Case (10) | | #1 | #2 | #3 | #4 | #1 | #2 | #3 | #4 | #1 | #2 | #3 | #4 |
| Component flow\Configuration | | — | — | — | — | A | A | A | A | B | B | B | B |
| | | | | | | | | | | (Note 1) | (Note 1) | (Note 1) | (Note 1) |
| H2 | kmol/h | 27 | 31 | 43 | 32 | 47 | 50 | 60 | 66 | 119 | 107 | 100 | 127 |
| CO | kmol/h | 40 | 41 | 43 | 62 | 20 | 21 | 25 | 28 | 40 | 40 | 42 | 62 |
| CO2 | kmol/h | 31 | 26 | 14 | 6 | 2 | 2 | 3 | 3 | 13 | 8 | 5 | 0.5 |
| N2 | kmol/h | 1 | 1 | 1 | 0 | 1 | 1 | 1 | 0 | 1 | 1 | 1 | 0.5 |
| S.N. = (H2 − | — | −0.06 | 0.08 | 0.51 | 0.38 | 2.04 | 2.04 | 2.04 | 2.04 | 2.04 | 2.04 | 2.04 | 2.04 |
| CO2)/(CO + CO2) | | | | | | | | | | | | | |
| Carbon Syngas | % | Ref. | Ref. | Ref. | Ref. | 31% | 35% | 49% | 45% | 73% | 73% | 83% | 92% |
| Recovery | | | | | | | | | | | | | |
| % Increase | % | — | — | — | — | Ref. | Ref. | Ref. | Ref. | 138% | 105% | 69% | 102% |
| Carbon Syngas | | | | | | | | | | | | | |
| Recovery | | | | | | | | | | | | | |

(Note 1)
Configuration B without the third recirculation loop (46), and thus without also the fourth mass transfer zone section 45 and flash steps 53

For methanol production, the balanced syngas would have an optimal stoichiometric ratio (SN) slightly above 2, as explain before. Although, it can also operate at lower or higher stoichiometric ratio. Other syngas conversion units or technology to obtain different end products may have different target stoichiometric ratio equations and/or values.

In an embodiment, the imported/external source of hydrogen 42 is from a renewable source and/or a source of low carbon intensity.

In an additional embodiment, the imported source of hydrogen 42 is from a water electrolysis with renewable power or low carbon intensity (CI) power, a biogas reforming or steam reforming, a low carbon intensity (CI) hydrogen source, or a low CI waste H₂ source.

Finally, the optimised carbon recovery balanced syngas 44 is feed to a syngas conversion unit 60 (syngas conversion reaction system), thus producing an optimised carbon content fuel and/or chemical as desired end product (61).

Considering that the downstream syngas conversion unit 60 will be designed with a maximum capacity and/or that the H₂ import 42 will also have a maximum supply capacity, to maximize plant profitability with varying scrubbed syngas composition and yield, design and operating handles have to be provided to maximize at all time the biofuel production and profitability.

At lower H₂+CO yield in the scrubbed syngas, higher AGR CO₂ recovery will be required to maintain the plant at is maximum methanol capacity and up to the availability of import hydrogen, since per reaction 3 more H₂ is required for conversion to methanol from CO₂.

At higher H₂+CO yield in the scrubbed syngas, lower AGR CO₂ recovery will be required to maintain the plant at is maximum methanol capacity, while minimizing H₂ import and thus variable operating costs.

In the provided AGR design, the AGR include the following design and operation handles to optimize the CO₂ recovery to maintain the plant at its maximum methanol capacity and optimal hydrogen usage.

The first 20 and second loops 22 flow rates and/or temperature are adjusted to achieve the target CO₂ content and low reduced sulfur content in the final clean syngas stream (40), and/or,

The pre-flash pressures of the first and/or second loops (21 and 23 respectively) are further optimised and the generated CO₂ streams (28 and 26 respectively) are recycle up-stream of the AGR to maximise CO₂ recovery in the final clean syngas stream (40).

The pressure of the first loop 20 low pressure flash 30 can be adjusted to optimize CO₂ recovery to achieve the exact required quantity of non-flammable rich CO₂ stream 32, and thus keeping the balance of the CO₂ in the clean syngas 40 leaving the AGR 12.

When high HCN and/or NH₃ removal level are required with FIG. 3 or 4 configurations, the AGR design further include the following design and operation handles to optimize the CO₂ recovery:
a third recirculation loop 46 flow rates and/or temperature are adjusted to achieve the target CO₂ content and low reduced nitrogen species content in the final clean syngas stream (40), and/or,
the pre-flash pressures of third loop (53) is further optimised and the generated CO₂ stream (47) is recycled up-stream of the AGR to maximise CO₂ recovery in the final clean syngas stream (40).

As exemplified herein, syngas production for methanol production have been used as an example. However, it can be applied with any syngas conversion process to chemical and fuel, that can be produced from H₂ and CO₂ in addition to H₂ and CO, i.e. Fischer Tropsch using iron based catalyst and ethanol using micro-organism bio-catalyst, etc. but not limited to. Desired Fischer Tropsch products, include fuels such as diesel, kerosene/jet fuel and/or naphtha following crude Fischer Tropsch products refining or upgrading, but also waxes, base oil, etc.

Methanol can also be used as an intermediate product. When first produced, it can be further converted to olefins (propylene and ethylene), formaldehyde, gasoline, naphtha, kerosene, aviation fuel, and/or diesel. Ethanol can be used as a fuel or as intermediate product, for example for ethylene production via dehydration.

In addition, the objective of maximizing syngas derived product yield with syngas CO₂ recovery and an external H₂ import from biomass rich feedstock, is also applicable to any carbonaceous feedstock with the objective to either maximise yield and production and/or reduce CO₂ green house gases (GHG) emissions. For example, plastic rich waste, waste rubber and tire, etc. chemical recycle via gasification process would also benefit of this novel AGR design by maximizing the total feedstock carbon conversion to the desired final product, thus minimizing the plant CO₂ GHG emission. More specifically, olefins production from methanol or ethanol can be used for plastic production, thus closing the circular loop when using plastic rich waste as gasification feedstock.

In an embodiment, the clean syngas stream 40 at the outlet of the AGR absorber achieves less than 100 ppbv, less than 10 ppbv, or alternatively less than 5 ppbv HCN and NH₃.

In another embodiment, the clean syngas stream 40 at the outlet of the AGR absorber achieves less than 10 ppmv, less than 5 ppmv, less than 1 ppmv, or alternatively less than 0.1 ppmv of combined sulfur species.

In another embodiment, the clean syngas stream at the outlet of the solid adsorbent bed 36 achieves less than 10 ppbv, or alternatively less than 5 ppbv of sulfur species, of halogen species (ex. HCl, HF, HBr, etc.), of arsine, and/or of metal (ex. Ni and/or Fe) carbonyl.

In another embodiment, the H₂S concentration in the clean syngas stream 40 at the outlet of the AGR absorber can be adjusted to achieves a specific higher desired concentration to meet the requirement of the downstream syngas conversion unit; while achieving low level of HCN and/or NH₃. This functionality is of special interest when using micro-organism syngas conversion technology (60) for which some H₂S is required to maintain the micro-organism activities, while HCN is a well-known poison. In such case, the H₂S concentration in the clean syngas stream 40 at the outlet of the AGR absorber can be maintained below 200 ppmv, alternatively below 100 ppmv, while achieving low level of HCN and/or NH₃. Reducing the H₂S removal requirement, allow to reduce the flowrate and/or stripping unit (25) duty of the second recirculation loop 22, reduce the inherent loss of CO₂ with the rich sulfur contaminants gas stream 29, thus increase the CO₂ recovery and concentration in the clean syngas 40 and producing a further optimised carbon content fuel and/or chemical as desired end product (61).

In another embodiment, this novel AGR design can also be used to fully removed the CO₂ from the scrubbed syngas 10 to produce an ultra-low CO₂ concentration clean syngas 40 or to achieve a specific lower CO₂ concentration in the clean syngas 40. With such objective, the recirculation loops pre-flash pressure are optimised to reduce CO₂ flash (and recycle back to the absorber), while still achieving the targe H₂ and CO recovery. The recirculation loops flow rate are increased to achieve higher CO₂ removal. Such flexible design is especially of interest for plant design when H₂ import is not available, and/or for syngas conversion technology that cannot make the desired end-product with CO₂+H₂.

In another embodiment, an external source of CO₂ or CO₂ input from another process effluent can be mixed with the clean syngas stream 40 along with the external source of hydrogen 42 to produce a balanced syngas 44 further boosted in carbon that meet the stoichiometric ratio target for the desired end product, thus further increasing the production of the desired end product. The flow of the external source of hydrogen (42) must be increased accordingly. Alternatively, if this additional CO₂ sources require cleaning, it could be fed to the AGR inlet.

Such CO₂ sources from another part of the process can be the unrecovered CO₂ lost in the rich sulfur stream (29) and/or nitrogen species rich stream (51). As such, to recover the CO₂ from this waste stream for catalytic synthesis, additional treatment units would be required to reduce contaminants down to ultra-low level. Such additional treatment units includes, but not limited to, additional absorption/stripping technology, solid adsorbent technologies, etc.

In another embodiment, additional ultra-clean high quality CO₂ can be recovered by adding a CO₂ recovery and purification unit (99) within the AGR as shown in FIG. 5.

In such configuration, the CO₂ loaded methanol (33), rather than being recycled to the AGR absorber (12), a portion (35) is sent to the top of a primary CO₂ column (70), which include at least one mass transfer zone. In addition, the CO₂ and sulfur species loaded methanol (24) is sent to the bottom of the primary CO₂ column (70). In the primary CO₂ column, the low sulfur CO₂ loaded methanol 35 acts as a scrubbing medium to keep the sulfur species down the column, while recovering a 2^{nd} medium quality CO₂ (71) from both loaded methanol streams. The primary CO₂ columns also remove additional non-condensable gas (ex. CO and H₂) from the bottom of the column, which further contributes to achieve ultra-low CO specifications in high quality CO₂ (stream 80).

The 1^{st} recovered medium quality CO₂ stream is in fact stream 32 as described earlier. Both medium quality CO₂ streams (32 and 71) are combined into stream 82, which meet the non-flammable quality criteria as described earlier. Optionally, they are compressed in a compressor (83) for use as inert gas in the plant front end feed system or other uses, as described earlier.

The bottom CO₂ and sulfur species loaded methanol (72) is fed to the secondary CO₂ column (73) in between its 2 mass transfer zones (74 and 75). The balance of the CO₂ loaded methanol (34), is fed to the top of the column and acts as a scrubbing medium to keep the sulfur species and other contaminants down the secondary CO₂ column (73), while recovering a high quality CO₂ stream (80) from both CO₂ and sulfur species loaded methanol (72) and the balance of the CO₂ loaded methanol (34). At the bottom of the secondary CO₂ column (73) the enriched sulfur species loaded methanol (76) is sent to a CO₂ flash unit (77) to maximize CO₂ recovery from the bottom enriched sulfur species loaded methanol (76).

The CO₂ flash unit (77) include a combination of low pressure flash with flash gas recompression and/or thermal heating assisted pressurized flash. The flash CO₂ rich streams (78) are returned at the bottom of the secondary CO₂ column (73).

The enriched sulfur species loaded methanol (79) is taken from the CO₂ flash unit (77) and then sent to the AGR stripping unit (25). The nitrogen species loaded methanol (48) is either sent directly to the AGR stripping unit (25) or could be incorporated into the CO₂ recovery and purification unit 99 to recover its small CO₂ content. The AGR stripping unit 25 regenerate both loaded methanol into a lean methanol (27), which is recycled at the top of the AGR absorber (12) section (18), as described earlier. The resulting AGR sulfur and/or nitrogen species waste gas stream (29) is thus further enriched in sulfur and nitrogen species, and thus results in lower loss of carbon (as CO₂) out of the process.

The recovered high quality CO₂ stream (80), is then, if required, compressed to higher pressure in a compressor (81). Depending on the required high quality CO₂ target specification it can then be treated in a solid phase absorbent unit (90) to remove remaining contaminants down to ultra-low ppm or ppb level as described before for the clean syngas guard beds (36). The produced ultraclean CO₂ stream (87, 88) can then be mixed with the clean syngas stream 40 along with the external source of hydrogen 42 to produce a balanced syngas 44 further boosted in carbon that meet the stoichiometric ratio target for the desired end product, thus further increasing the production of the desired end product. The flow of the external source of hydrogen (42) must be increased accordingly, as described earlier.

Table 4 below show that additional CO₂ recovery and yield increase with this additional functionality to the AGR.

TABLE 4
Improved process scrubbed syngas carbon recovery with CO2 Recovery and Purification
unit and with H₂ import compared to traditional approach with WGS.

| | | Scrubbed | | Traditional | | | | Improved Process | |
|---|---|---|---|---|---|---|---|---|---|
| | | Syngas (10) | | Approach | | Improved Process | | with CO2 Recovery and | |
| | | (Total 100 | | with WGS - | | with H2 Import - | | Purification Unit (99) - | |
| | | kmol/h basis) | | Balanced Syngas | | Balanced Syngas (44) | | Balanced Syngas (44) | |
| Scrubbed Syngas Case (10) | | #1 | #2 | #1 | #2 | #1 | #2 | #1 | #2 |
| Component flow\Configuration. | | — | — | A | A | B | B | C | C |
| | | | | | | (Note 1) | (Note 1) | (Note 1, 2) | (Note 1, 2) |
| H2 | kmol/h | 27 | 31 | 47 | 50 | 119 | 107 | 147 | 128 |
| CO | kmol/h | 40 | 41 | 20 | 21 | 40 | 40 | 40 | 40 |
| CO2 | kmol/h | 31 | 26 | 2 | 2 | 13 | 8 | 22 | 15 |
| N2 | kmol/h | 1 | 1 | 1 | 1 | 1 | 1 | 1 | 1 |
| S.N. = (H2 − | — | −0.06 | 0.08 | 2.04 | 2.04 | 2.04 | 2.04 | 2.04 | 2.04 |
| CO2)/(CO + CO2) | | | | | | | | | |
| Carbon Syngas | % | Ref. | Ref. | 31% | 35% | 73% | 73% | 86% | 83% |
| Recovery | | | | | | | | | |
| % Increase | % | — | — | Ref. | Ref. | 138% | 105% | 178% | 134% |
| Carbon Syngas | | | | | | | | | |
| Recovery | | | | | | | | | |

(Note 1)
Configuration B and C without the third recirculation loop (46), and thus without also the fourth mass transfer zone section 45 and flash steps 53
(Note 2)
Configuration C without optional first solvent circulation loop flash steps 30

For sure, such additional CO₂ recovery and desired product yield increase require availability for additional imported hydrogen (42). For different reasons, a specific project and plant location may have limited access to imported hydrogen. As such, one of the important feature of this invention is that each project can decide to include all or only part of the described features herein to achieve the specific requirements of the project and minimize the project profitability and/or environmental foot print.

If lower quality CO₂ are required, the flash step 30 can be omitted, and thus first loop 31 would be split in 2 (34 and 35), rather than stream 33, to feed the primary (70) and secondary (73) CO₂ columns. In that configuration, all the medium CO₂ quality would be supplied from the primary CO₂ column via stream 71. Alternatively, the primary CO₂ column could be omitted if lower CO₂ quality is required. In that configuration, the CO₂ loaded methanol (33) is fed directly at the top of the secondary CO₂ columns (73), and the CO₂ and sulfur species loaded methanol (24) is fed directly to the secondary CO₂ column (73) in between its 2 mass transfer zones (74 and 75).

In another embodiment, if limited hydrogen supply is available, or the desired end product synthesis catalyst and unit (60) cannot convert the recovered ultraclean CO₂ (87) into additional desired product, the ultraclean CO₂ can be sold off-site or send for storage (i.e. CCS) (stream 89).

Alternatively, when the desired end product synthesis catalyst and unit (60) cannot convert the recovered ultraclean CO₂ (87) into additional desired product, the ultraclean CO₂ could be sent (stream 89) to a methanol reactor along with additional imported hydrogen to co-produce methanol in the plant. Alternative to methanol co-production are any technology that can convert CO₂+H₂ to a desired end product, as described before.

In another embodiment, any excess (85) of medium quality CO₂ (84) could be mixed with the high quality CO₂ (86), to further maximize desired end-product yield and production.

When a CO₂ recovery and purification unit (99) is included in the AGR configurations, the AGR design further include the following design and operation handles to optimize the CO₂ recovery from the variable scrubbed syngas source:
The first 20 and second loops 22 flow rates and/or temperature are adjusted to achieve the target CO₂ content and low sulfur content in the final clean syngas stream (40), and/or, the target recovery of high purity CO₂ (87).
The pre-flash pressures of the first and/or second loops (21 and 23 respectively) are further optimised and the generated CO₂ streams (28 and 26 respectively) are recycle up-stream of the AGR to balance the desired CO₂ recovery in the final clean syngas stream (40) versus the recovery of high purity and/or ultraclean CO₂ (87).
The pressure of the first loop 20 and low pressure flash 30 can be used to reduce the CO content in the high quality CO₂ (80).
The CO₂ flash unit (77) pressure and/or temperature can be adjusted to achieve the desired high purity CO₂ (87).

In another embodiment, the AGR stripping unit (25) is a split loading nitrogen and sulfur species stripping unit design, rather than a standard configuration stripper where the combined contaminants loaded solvents are sent at the top of the stripper to be stripped off their contaminants as the solvents flow down to the bottom of the stripper to generate a regenerated solvent at the bottom of the stripper.

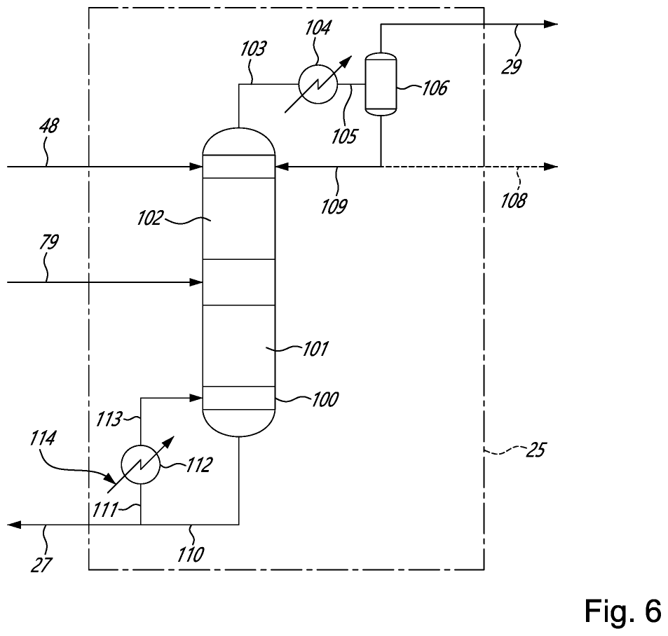

The split loading design option (FIG. 6) of the AGR stripping unit (25) allow to further improve the overall energy efficiency of the overall AGR unit as shown in Table 5 below.

TABLE 5
AGR efficiency improvement with split loading stripper
design compared to standard configuration stripper

| | | Standard | Split Loading |
|---|---|---|---|
| | Unit/ | Stripper (25) | Stripper (100) |
| | Case | #1 (Note 1) | #2 (Note 1) |
| Nb of Theoritical Stages (single | Nb | 18 | — |
| mass transfer zone) | | | |
| Nb of Theoritical Stages - Bottom | Nb | — | 6 |
| mass transfer zone (101) | | | |
| Nb of Theoritical Stages - Bottom | Nb | — | 12 |
| mass transfer zone (102) | | | |
| Total Nb of Theoritical Stages | Nb | 18 | 18 |
| in stripper | | | |
| Heat Duty (25) | kW | 2471 | — |
| Heat Duty (52) | kW | — | — |
| Heat Duty (100) | kW | — | 2471 |
| Total Heat duty | kW | 2471 | 2471 |
| AGR H2S removal efficiency | % | >99.9 | >99.9 |
| AGR HCN removal efficiency | % | 98.5 | >99.9 |

(Note 1):
Both cases include the third recirculation loop (46), and thus also the fourth mass transfer zone section 45 and flash steps 53

In the split loading design option, the stripper column (100) has at least 2 mass transfer zone sections (101 and 102). The nitrogen species loaded methanol (48) is fed at the top of the stripper column and above the top mass transfer zone (102), while the sulfur species loaded methanol (79) is fed in the middle of the stripper column (100) in between the top (102) and bottom (101) mass transfer zone sections. Thermal energy (114) is supplied to the stripper column (100) via the stripper reboiler (112), which evaporate a portion (111) of the column bottom product (110) to generate the vapor boil-up (113), which is returned to the bottom of the stripper column (100). The stripper column (100) is also equipped with a condenser (104) which cool and condense the methanol vapor in the column overhead vapor (103). At the outlet of the condenser (104) stream 105 contains the condensed methanol and non-condensable vent gases, which are separated in the reflux drum (106) into the liquid reflux (109) required for the column operation, and the sulfur and/or nitrogen species vent gases (29). The regenerated solvent (27) at the bottom of the stripper column (100) is then recycled, as described earlier, back to the absorber column (12) top mass transfer zone section (18).

In an embodiment, when the scrubbed syngas (10) contain significant concentration of aromatic such as benzene and toluene, a liquid purge (108) may be extracted from the reflux steam (109) to minimise build-up to these aromatic compound in the AGR loops.

In an embodiment, this novel AGR design, can also be integrated with processes incorporating reverse water gas shift (RWGS) unit to convert the recovered CO₂ along with the imported H₂ to generate additional carbon monoxide prior to the syngas conversion unit, as described in patent application U.S. 63/185,482, the content of which is incorporated herein in its entirety. The additional CO production being mixed with the balance of the clean syngas to generate a boosted CO clean syngas.

In another embodiment, as described in patent application U.S. 63/185,482, the recovered CO₂ of this novel AGR design can be recycled back to the carbonaceous feedstock gasification and/or reforming unit to reduce the reformed syngas H₂/CO ratio, increase the total CO yield and production, and thus generating a boosted CO scrubbed syngas and a boosted CO clean syngas at the outlet of the AGR.

In both cases, the boosted CO clean syngas is mixed with hydrogen producing a balanced syngas stream, wherein the balanced syngas stream meet the stoichiometric ratio requirement of the syngas conversion unit. Such option being advantageous when the syngas conversion unit can only convert in-situ H₂+CO and not H₂+CO₂ into desired product, for example but not limited to Fischer Tropsch unit or technology using Cobalt based catalyst, or ethanol production technology using the methanol carbonylation pathway

While the disclosure has been described with particular reference to the illustrated embodiment, it will be understood that numerous modifications thereto will appear to those skilled in the art. Accordingly, the above description and accompanying drawings should be taken as illustrative and not in a limiting sense. For simplification purpose, several secondary details were omitted, such as pump, heat exchangers, some compressors, etc.

While the disclosure has been described in connection with specific embodiments thereof, it will be understood that it is capable of further modifications and this application is intended to cover any variations, uses, or adaptations and including such departures as come within known or customary practice within the art and as may be applied to the essential features hereinbefore set forth, and as follows in the scope of the appended claims.

1. A process for optimizing syngas carbon utilisation, syngas purity for subsequent syngas conversion into a downstream syngas conversion unit from a scrubbed syngas source comprising the steps of:
a) feeding a scrubbed syngas into a purification unit comprising at least one absorption unit to partially remove CO₂ from said scrubbed syngas, producing a clean syngas stream adjusted to optimal target carbon content and a CO₂ rich stream, wherein the purification unit is an acid gas removal unit (AGR), wherein in said AGR,
CO₂ and sulfur species are absorbed from the scrubbed syngas in a absorption unit using said selective solvent, producing a loaded solvent and a clean syngas stream;
the loaded solvent is withdrawn through at least one recirculation loop;
the loaded solvent is pre-flashed at an intermediate pressure to recover absorbed H₂ and CO, producing the CO₂ rich stream rich in H₂ and CO and a flashed solvent stream;
the flashed solvent is flashed at lower pressure to recover CO₂, generating a non-flammable CO₂ rich stream and a second flashed solvent; and
the second flashed solvent is stripped in a first stripping unit to remove the sulfur species, producing a rich sulfur species stream and a clean solvent which is recycled back into the absorption unit;
b) mixing the clean syngas stream with hydrogen producing a balanced syngas stream, wherein the balanced syngas stream meet the stoichiometric ratio requirement of the syngas conversion unit; and
c) feeding said balanced syngas stream in said syngas conversion unit.

2. The process of claim 1, wherein the at least one absorption unit further removes sulfur species and produces an additional sulfur species rich stream.

3. The process of claim 2, wherein the sulfur species is H₂S, COS, CS₂, or a combination thereof.

4. The process of claim 1, wherein the at least one absorption unit further removes nitrogen contaminant species and produces an additional nitrogen contaminant rich stream.

5. The process of claim 1, wherein the scrubbed syngas source comprises between 25 to 45 mol % of H₂, 30 to 65 mol % of CO and 6 to 40 mol % of CO₂ and/or wherein the clean syngas stream composition comprises 30% to 50 mol % H₂, 40 to 68 mol % CO and 0 to 25 mol % CO₂.

6. (canceled)

7. The process of claim 1, further comprising a step of recycling the CO₂ rich stream for use as inerting gas, exporting said CO₂ rich stream for Carbon Capture and Storage (CCS), and/or producing saleable merchant CO₂ from said CO₂ rich stream.

8. The process of claim 1, wherein a first portion of the loaded solvent is withdrawn through a first recirculation loop and a second portion of the loaded solvent is withdrawn through a second recirculation loop, and where,
the first portion of the loaded solvent is pre-flashed at an intermediate pressure recovering the H₂ and CO contained in the loaded solvent, producing a first CO₂ stream rich in H₂ and CO and a first flashed solvent, and the second portion of the loaded solvent is pre-flashed at an intermediate pressure recovering H₂ and CO contained in the loaded solvent, producing a second CO₂ stream rich in H₂ and CO and a second flashed solvent;
the first flashed solvent is flashed at lower pressure generating a non-flammable CO₂ rich stream and a clean solvent which is recycled back into the absorption unit;
the first and second CO₂ streams rich in H₂ and CO are recycled in the scrubbed syngas stream upstream of the absorption unit; and
the second solvent is stripped in a first stripping unit to remove sulfur species, producing a rich sulfur species stream and a clean solvent which is recycled back into the absorption unit.

9. The process of claim 1, wherein the selective solvent of the purification unit further removes the sulfur species, and nitrogen species, producing a rich sulfur and nitrogen species stream.

10. The process of claim 9, wherein a first portion of the loaded solvent is withdrawn through a first recirculation loop, a second portion of loaded solvent is withdrawn through a second recirculation loop, and a third portion of loaded solvent is withdrawn through a third recirculation loop where;
the first portion of the loaded solvent is pre-flashed at an intermediate pressure recovering the H₂ and CO contained in the loaded solvent, producing a first CO₂ stream rich in H₂ and CO and a first flashed solvent; and the second portion of the loaded solvent is pre-flashed at an intermediate pressure recovering H₂ and CO contained in the loaded solvent, producing a second CO₂ stream rich in H₂ and CO and a second flashed solvent; and, optionally, the third portion of the loaded solvent is pre-flashed at an intermediate pressure recovering the H₂ and CO contained in the loaded solvent, producing a third CO₂ stream rich in H₂ and CO and a third flashed solvent;
the first flashed solvent is flashed at lower pressure generating a non-flammable CO₂ rich stream and a clean solvent which is recycled back into the absorption unit;
the first, second and optional third CO₂ streams rich in H₂ and CO are recycled in the scrubbed syngas stream upstream of the absorption unit; and
the second flashed and third flashed solvents, or unflashed solvents are stripped in a stripping unit to remove the sulfur species, and nitrogen species, producing a rich sulfur and nitrogen species stream and a clean solvent which is recycled back into the absorption unit.

11-19. (canceled)

20. The process of claim 1, wherein the non-flammable CO₂ rich stream is further used as inerting gas, recovered for Carbon Capture and Storage (CCS) and/or to produce saleable merchant CO₂.

21. The process of claim 1, further comprising treating the clean syngas stream with at least one solid adsorbent bed before or after mixing the clean syngas stream with hydrogen.

22-29. (canceled)

30. The process of claim 1, wherein H₂S concentration in the clean syngas stream is adjusted to achieves a specific desired concentration, to meet the requirement of the downstream syngas conversion unit; while achieving low level of HCN and/or NH₃ concentration.

31-32. (canceled)

33. The process of claim 1, wherein the hydrogen is imported from an external source.

34-35. (canceled)

36. The process of claim 1, wherein the purification unit comprises chilled methanol as a solvent.

37. The process of claim 1, wherein the balanced syngas stream meet the stoichiometric ratio requirement of the syngas conversion unit to produce fuel, a chemical, or a Fischer Tropsch product.

38-39. (canceled)

40. The process of claim 1, wherein the scrubbed syngas is from a gasification and/or reforming of a carbonaceous material.

41-44. (canceled)

45. The process of claim 1, further comprising admixing an external source of CO₂ or CO₂ input from another process effluent into the clean syngas stream along with the external source of hydrogen to produce a balanced syngas further boosted in carbon that meet the stoichiometric ratio of the desired end product, thus further increasing the production of the desired end product.

46-53. (canceled)

54. The process of claim 1, further comprising a reverse water gas shift (RWGS) unit to convert a portion of the CO₂ along with a portion of the hydrogen to generate additional carbon monoxide prior to the syngas conversion unit.

55. (canceled)

56. The process of claim 1, wherein CO₂ is recycled back to a carbonaceous feedstock gasification and/or reforming unit to reduce the scrubbed syngas source H₂/CO ratio, increasing a total CO yield and production, and generating a boosted CO scrubbed syngas and a boosted CO clean syngas.

57. (canceled)

58. The process of claim 1, wherein the purification unit comprises a stripping unit including a split loading stripper column with at least a top and bottom mass transfer zone sections, where nitrogen species loaded methanol is fed at the top of the stripper column and above the top mass transfer zone, while the sulfur species loaded methanol is fed in the middle of the stripper column in between the top and bottom mass transfer zone sections.

59-60. (canceled)
